(19) United States
(12) Patent Application Publication
(10) Pub. No.: US 2025/0059230 A1
(43) Pub. Date: Feb. 20, 2025

(54) AN IMPROVED METHOD FOR SEPARATION OF LOW MOLECULAR WEIGHT PROTEINS

(71) Applicant: Kashiv BioSciences, LLC, Piscataway, NJ (US)

(72) Inventors: Roshan Ganeshlal Upadhyay, Ahmedabad (IN); Jaykumar Rameshchandra Kardani, Ahmedabad (IN); Aakansha Shah, Ahmedabad (IN)

(21) Appl. No.: 18/720,877

(22) PCT Filed: Dec. 17, 2022
(86) PCT No.: PCT/IB2022/062426

§ 371 Date: Jun. 17, 2024

(30) Foreign Application Priority Data
Dec. 17, 2021 (IN) 202121058987

(51) Int. Cl. C07K 1/26; C07K 16/46

(52) U.S. Cl. CPC C07K 1/26; C07K 16/46; C07K 2319/30

(57) ABSTRACT

The present invention provides the method for the separation of at least more than two Low molecular weight (LMWs) impurities present in the protein mixture comprising fusion protein CTL4-IgG1 by using Capillary Electrophoresis-Sodium dodecyl sulfate (CE-SDS) wherein the loading amount of protein mixture is more than 270 μg. The invention further provides a ratio of protein to sodium dodecyl sulfate is 2.7:1 or higher.

### FIELD OF THE INVENTION

The present invention provides the method for the separation of at least more than two Low molecular weight (LMWs) impurities present in the protein mixture comprising fusion protein CTL4-IgG1 by using Capillary Electrophoresis-Sodium dodecyl sulfate (CE-SDS) wherein the loading amount of protein mixture is more than 270 μg. The invention further provides a ratio of protein to sodium dodecyl sulfate is 2.7:1 or higher.

### BACKGROUND OF THE INVENTION

Fusion proteins are very complex proteins and preparation of biosimilars, or biological identical molecules are very challenging as it is mandatory to match impurity profile such as LMW to comply with regulatory bodies. The one of the challenging tasks is to separate all LMWs present in the protein mixture. If it is not separated properly, it may affect the protein quality and make downstream process lengthy and expensive. To tackle the quality issue, a combination of multiple analytical approaches is necessary. The present invention provides a separation method which is reproducible and helpful in determining batch to batch consistency. Capillary SDS gel electrophoresis (CE-SDS) of proteins is a well-accepted separation method in the biomedical and biopharmaceutical industry. Electric field mediated separation methods of biologically important polymers, such as nucleic acids and SDS protein complexes, have long been routinely applied for molecular size estimation and purity analysis. In the present invention, while performing CE-SDS to separate and quantify impurities such as LMWs in the protein mixture, the amount of protein is increased at suitable ratio of SDS during sample preparation, which gives more peak of LMWs during separation as compared to the sample having lower amount of protein less than 270 μg.

### SUMMARY OF THE INVENTION

In an embodiment, the invention provides the process for performing Capillary Electrophoresis-Sodium dodecyl sulfate (CE-SDS) to separate and quantify impurities at least more than two Low molecular weight impurities (LMWs) present in protein mixture comprising fusion protein, wherein the loading amount of protein mixture is more than 270 μg.

In certain embodiment, the present invention load protein sample about 270 μg to separate at least three peaks of LMW in CE-SDS.

In certain embodiment, the present invention load protein sample about 360 μg to separate at least three peaks of LMW in CE-SDS.

In certain embodiment, the present invention load protein sample about 360 μg to separate at least four peaks of LMW in CE-SDS.

In certain embodiment, the present invention load protein sample about 450 μg to separate at least four peaks of LMW in CE-SDS.

In certain embodiment, the present invention load protein sample about 450 μg to separate at least five peaks of LMW in CE-SDS.

In certain embodiment, the protein mixture is obtained from harvest. In another certain embodiment, the protein mixture is obtained from one or more chromatographic steps. In certain embodiment, the present invention load protein sample about 450 μg to separate at least five peaks of LMW in CE-SDS.

In an embodiment, the improved process for the separation of more than two low molecular weight proteins presents in the protein mixture comprising;
a) protein mixture comprising at least the impurity and the protein of interest, wherein the protein mixture does not contain a full-length antibody having molecular weight about 150 kDa;
b) mixing the protein mixture at a suitable ratio with sodium dodecyl sulphate or SDS to form treated protein mixture;
c) optionally incorporating a 10 kDa marker into the treated protein mixture;
d) injecting the treated protein mixture in CE-instrument;
e) performing CE-SDS by injecting the treated protein mixture in a CE-instrument; and
f) separating more than two low molecular weight proteins;
   wherein the treated protein mixture comprises protein to SDS ratio is higher than 2.7:1.

In an embodiment, the protein of interest is a fusion protein.

In another embodiment, the fusion protein is CTLA4-IgG having molecular weight less than 100 kDa.

In certain embodiment, the composition of protein mixture comprising;
a) CTLA4-IgG fusion protein;
b) low molecular weight impurities comprises;
   i) first LMW about 80 kDa;
   ii) second LMW about 45 kDa;
   iii) third and fourth LMW about 25 kDa to 30 kDa;
      wherein the first, second, third and fourth LMWs are separated by CE-SDS.

In an embodiment, the treated protein mixture comprises protein to SDS ratio selected from about 3:1, about 3.6:1, about 4.0:1, about 4.5:1, and about 5:1.

In an embodiment, the protein concentration in treated protein mixture is selected from about 3mg/ml, about 3.5 mg/ml, about 4.0 mg/ml, and about 4.5 mg/ml.

In an embodiment, the separation and quantification of more than two low molecular weight impurity is improved compared to CE-SDS performed at protein concentration below 2.7 mg/ml.

In an embodiment, the separation and quantification of more than two low molecular weight impurity is improved compared to CE-SDS performed at protein concentration below 3.6 mg/ml.

In an embodiment, the separation and quantification of more than two low molecular weight impurity is improved compared to CE-SDS performed at protein concentration below 4.5 mg/ml.

In an embodiment, the separation and quantification of low molecular weight impurity is performed at 25° C.

In an embodiment, the CE-SDS is performed at suitable separation voltage to separate more than two low molecular weight protein in less than 22 minutes.

In an embodiment, the CE-SDS is performed at suitable separation voltage to separate four low molecular weight protein in less than 22 minutes, preferably less than 20 minutes.

In an embodiment, the protein mixture is obtained from harvest or post affinity chromatography or post final purification steps.

In an embodiment, the present invention provides an improved method for separation and quantification of impurities in a protein sample comprising;
a) obtaining a protein mixture from partially or completely purification comprising protein of interest and LMWs;
b) determine the amount of protein mixture about 270 μg;
c) mixing the protein mixture at a suitable ratio with sodium dodecyl sulphate or SDS to form treated protein mixture;
d) loading the treated protein mixture to said Capillary Electrophoresis-Sodium dodecyl sulfate (CE-SDS) capillary;
e) applying the suitable voltage to CE-SDS instrument;
f) separated and quantified the LMW species present in the treated protein mixture;
   wherein, separation and quantification of LMW is improved when protein amount loaded higher than 270 μg.

In an embodiment, the present invention provides an improved method for separation and quantification of impurities in a protein sample comprising;
a) obtaining a protein mixture from partially or completely purification comprising protein of interest and LMWs;
b) determine the amount of protein mixture about 360 μg;
c) mixing the protein mixture at a suitable ratio with sodium dodecyl sulphate or SDS to form treated protein mixture;
d) loading the treated protein mixture to said Capillary Electrophoresis-Sodium dodecyl sulfate (CE-SDS) capillary;
e) applying the suitable voltage to CE-SDS instrument;
f) separated and quantified the LMW species present in the treated protein mixture;
   wherein, separation and quantification of LMW is improved when protein amount loaded about or higher than 360 μg.

In an embodiment, the present invention provides an improved method for separation and quantification of impurities in a protein sample comprising;
a) obtaining a protein mixture from partially or completely purification comprising protein of interest and LMWs;
b) determine the amount of protein mixture about 360 μg;
c) mixing the protein mixture at a suitable ratio with sodium dodecyl sulphate or SDS to form treated protein mixture;
d) loading the treated protein mixture to said Capillary Electrophoresis-Sodium dodecyl sulfate (CE-SDS) capillary;
e) applying the suitable voltage to CE-SDS instrument;
f) separated and quantified the LMW species present in the treated protein mixture;
   wherein, separation and quantification of LMW is improved when protein amount loaded about or higher than 360 μg.

In an embodiment, the present invention provides an improved method for separation and quantification of impurities in a protein sample comprising;
a) obtaining a protein mixture from partially or completely purification comprising protein of interest and LMWs;
b) determine the amount of protein mixture about 450 μg;
c) mixing the protein mixture at a suitable ratio with sodium dodecyl sulphate or SDS to form treated protein mixture;
d) loading the treated protein mixture to said Capillary Electrophoresis-Sodium dodecyl sulfate (CE-SDS) capillary;
e) applying the suitable voltage to CE-SDS instrument;
f) separated and quantified the LMW species present in the treated protein mixture;
   wherein, separation and quantification of LMW is improved when protein amount loaded about or higher than 450 μg.

In an embodiment, the present invention provides an improved method for separation and quantification of impurities in a protein sample comprising;
a) obtaining a protein mixture from partially or completely purification comprising protein of interest and LMWs;
b) determine the amount of protein mixture about 450 μg;
c) mixing the protein mixture at a suitable ratio with sodium dodecyl sulphate or SDS to form treated protein mixture;
d) loading the treated protein mixture to said Capillary Electrophoresis-Sodium dodecyl sulfate (CE-SDS) capillary;
e) applying the suitable voltage to CE-SDS instrument;
f) separated and quantified the LMW species present in the treated protein mixture;
   wherein, separation and quantification of LMW is improved when protein amount loaded about or higher than 450 μg.

In an embodiment, the present invention provides an improved method for separation and quantification of impurities in a protein sample comprising;
a) obtaining a protein mixture from partially or completely purification comprising protein of interest and LMWs;
b) determine the amount of protein mixture about 450 μg;
c) mixing the protein mixture at a suitable ratio with sodium dodecyl sulphate or SDS to form treated protein mixture;
d) loading the treated protein mixture to said Capillary Electrophoresis-Sodium dodecyl sulfate (CE-SDS) capillary;
e) applying the suitable voltage to CE-SDS instrument;
f) separated and quantified the LMW species present in the treated protein mixture;
   wherein, separation and quantification of LMW is improved when protein amount loaded about or higher than 450 μg.

In an embodiment, the present invention provides an improved method for separation and quantification of impurities in a protein sample comprising;
a) obtaining a protein mixture from partially or completely purification comprising protein of interest and LMWs;
b) determine the amount of protein mixture about 450 μg;
c) mixing the protein mixture at a suitable ratio with sodium dodecyl sulphate or SDS to form treated protein mixture;
d) loading the treated protein mixture to said Capillary Electrophoresis-Sodium dodecyl sulfate (CE-SDS) capillary;
e) applying the suitable voltage to CE-SDS instrument;
f) separated and quantified the LMW species present in the treated protein mixture;
   wherein, separation and quantification of LMW is improved when protein amount loaded about or higher than 450 μg.

### BRIEF DESCRIPTION OF FIGURES

FIG. 1: shows comparison between the electropherograms of sample (Abatacept) (90 μg and 450 μg) after increasing protein amount during sample preparation.

FIG. 2: Electropherograms showing five LMW peaks of Abatacept sample (450 μg) after increasing protein amount during sample preparation.

### DETAIL DESCRIPTION OF THE INVENTION

The present invention relates to an improved method for quantification of impurities of protein mixture comprises of at least one antibody or fusion protein, wherein the analysis of protein mixtures is performed with Capillary Electrophoresis-Sodium dodecyl sulfate (CE-SDS).

The term “Capillary Electrophoresis-Sodium dodecyl sulfate (CE-SDS)” or “CE-instrument” refers to electrophoresis process based on migration of SDS bound proteins through a gel-filled capillary in an electric field. CE-SDS is a technique employed to quantify the low molecular weight (LMW) impurities of proteins. SDS binds to the proteins in a constant ratio such that the magnitude of the charge of each species is directly proportional to its molecular weight. Analytes are introduced to the gel-filled capillary and migrate from the cathode (negative) towards the anode (positive). Small molecules can move through the gel matrix more rapidly than large molecules; therefore, the analytes are detected in order of increasing molecular size. CE-SDS either in reduced (rCE-SDS) or non-reduced (nrCE-SDS) form, is widely used for purity evaluation and impurity analysis of monoclonal antibody (mAb) drugs.

The term “non-reduced condition” refers to the separation of protein subunits that are not linked by disulfide bonds (i.e., noncovalently linked proteins). For the non-reduced analysis, the native protein is treated with SDS prior to separation to mask the protein native charges. For reduced analysis, the sample is treated with SDS, and either dithiothreitol (DDT) or beta-mercaptoethanol (BME) to reduce the native protein structure.

The term “capillary” refers to the capillary tube or cartridge of Capillary Electrophoresis-Sodium dodecyl sulfate (CE-SDS) which is made of fused silica and is sometimes coated with polyimide. Each side of the capillary tube is dipped in a vial containing the electrode and an electrolytic solution, or aqueous buffer. There is usually a small window near the cathodic end of the capillary which allows UV-VIS light to pass through the analyte and measure the absorbance. A photomultiplier tube is also connected at the cathodic end of the capillary, which enables the construction of a mass spectrum, providing information about the mass to charge ratio of the ionic species.

The term “SDS” refers to Sodium dodecyl sulfate. SDS is a detergent with a strong protein-denaturing effect and binds to the protein backbone at a constant molar ratio. In the presence of SDS and a reducing agent that cleaves disulfide bonds critical for proper folding, proteins unfold into linear chains with negative charge proportional to the polypeptide chain length. SDS is commonly used to obtain high resolution separation of complex mixtures of proteins. The method initially denatures the proteins that will undergo electrophoresis.

The term “about”, as used herein, is intended to refer to ranges of approximately 10-20% greater than or less than the referenced value. In certain circumstances, one of skill in the art will recognize that, due to the nature of the referenced value, the term “about” can mean more or less than a 10-20% deviation from that value.

In an embodiment, 270 μg of protein should be 297 μg or more, 360 μg of protein should be about 324 μg to 396 μg and 450 μg of protein should be 405 μg to 495 μg which is 10% deviation from the actual value.

The term “comprises” or “comprising” is used in the present description, it does not exclude other elements or steps. For the purpose of the present invention, the term “consisting of” is considered to be an optional embodiment of the term “comprising of”. If hereinafter a group is defined to comprise at least a certain number of embodiments, this is also to be understood to disclose a group which optionally consists only of these embodiments.

The term “CTLA4-IgG1” or “Abatacept” used herein refers to a recombinant DNA generated fusion protein used to treat the symptoms of rheumatoid arthritis and to prevent joint damage caused by these conditions. Abatacept is a biological product developed for immunosuppression by blocking T cell activation through inhibition of costimulatory signals and is indicated for treatment of rheumatoid arthritis. Abatacept is a soluble homodimeric fusion protein of two identical subunits covalently linked by one disulfide bond. Each subunit consists of the modified amino acid sequence of the human cytotoxic lymphocyte associated antigen 4 (CTLA4), human immunoglobin IgG1 hinge, CH2 and CH3 region (Fc). Modification to the original sequences were introduced to avoid unintended disulfide bond formation and to reduce the ability of complement activation.

The term used “low molecular weight” or “LMW” or “first LMW” or “second LMW” or “third LMW” or “fourth LMW” or “fifth LMW” species which are protein backbone-truncated fragments & considered as product-related impurities that contribute to the size heterogeneity of fusion protein. LMW species often have low or substantially reduced activity relative to the monomeric form of the fusion protein and can lead to immunogenicity or potentially impact pharmacokinetic properties in vivo. As a result, LMW species are considered critical quality attributes that are routinely monitored during drug development and as part of release testing of purified drug product during manufacturing.

The term “electropherograms” refers to a plot of sequence data obtained via electrophoresis.

The term “protein sample” or “protein mixture” refers to the sample or mixture comprising at least one fusion protein and size variants impurities.

The term “protein mixture” and “protein sample” are interchangeable respectively in the present invention.

The term “treated protein mixture” refers to the protein sample treated with sodium dodecyl sulfate or SDS at a suitable protein to SDS ratio which 2.7:1 or higher.

The term “amount of protein” refers to the concentration or quantity of protein in the sample used while sample preparation. The term “determine the amount of protein mixture” refers to the calculating the protein amount to be added for the protein sample mixture preparation.

The term “sample preparation” refers to the preparation of protein sample where protein is about three times or higher than the SDS quantity. Usually, the ratio of protein and SDS recommended and available in the arts is about 0.9:1 or 1:1. In the present disclosure provides a improved method comprising the ratio of protein to SDS is 2.7:1 or higher. The skilled person does not have reasonable expectation of success with respect to increasing the amount of CTLA4-IgG1 protein because he would have thought about main peak split of protein due to high protein concentration and low SDS amount and further he does not even observe LMW peaks when he increases the CTLA4-IgG1 protein concentration at least by 2 fold. Furthermore, the arts available does not motivate towards using higher protein to SDS ratio.

In certain embodiment, the amount of protein used in sample preparation is selected from about 270 μg, about 300 μg, about 330 μg, about 360 μg, about 370 μg, about 380 μg, about 390 μg, about 400 μg, about 410 μg, about 420 μg, about 430 μg, about 440 μg, about 450 μg, about 460 μg, about 470 μg, about 480 μg, about 490 μg, about 500 μg, about 540 μg, about 630 μg, about 720 μg, about 810 μg, about 900 μg, about 910 μg about 920 μg about 925 μg, about 950 μg, about 960 μg, about 970 μg, about 980 μg, and about 1000 μg for separation and quantification of impurities in a protein sample.

In preferred embodiment, the amount of protein used in sample preparation is selected from about 450 μg.

The term “buffer” used herein refers to the solution in which the sample will be separated. The buffer used herein is SDS-MW Sample Buffer. The SDS-MW Sample Buffer is provided as part of the SDS-MW Analysis Kit. This buffer consists of 100 mM Tris-HCl at pH 9.0 with 1% SDS.

In an embodiment, the amount of buffer used herein is selected from about 15 μL, about 25 μL, about 35 μL, about 45 μL, about 55 μL, about 65 μL, about 75 μL, about 85 μL, about 95 μL, about 105 μL, about 115 μL, about 125 μL, about 135 μL, about 145 μL, about 155 μL, about 165 μL, about 175 μL, about 185 μL, about 195 μL, about 205 μL, about 215 μL, and about 225 μL.

In another embodiment, the amount of buffer used herein is selected from about 10 μL, about 50 μL, about 100 μL, about 150 μL, about 200 μL, about 250 μL, and about 300 μL.

In preferred embodiment, the amount of buffer used herein is selected from about 100 μL.

A 10 kDa protein Internal Standard used herein is used as a mobility marker. The mobility of all protein samples is calculated relative to this mobility marker allowing for more accurate size estimation and analyte identification.

In an embodiment, the amount of 10 kDa protein Internal Standard used herein is selected from about 1 μL, about 2 μL, about 3 μL, about 4 μL, about 5 μL, about 6 μL, about 7 μL, about 8 μL, about 9 μL, and about 10 μL.

In preferred embodiment, the amount of 10 kDa protein Internal Standard used herein is selected from about 2 μL.

The term “IAM” refers to iodoacetamide solution. The IAM (iodoacetamide) solution acts as the alkylating agent during preparation of the sample to minimize any heterogeneity created from partial auto-reduction of the protein.

In an embodiment, the concentration of IAM solution used herein is selected from about 10 mM. about 25 mM, about 50 mM, about 75 mM, about 100 mM, about 125 mM, about 150 mM, about 175 mM, about 200 mM, about 225 mM, about 250 mM, about 275 mM, and about 300 mM.

In preferred embodiment, the concentration of IAM solution used herein is selected from about 250 mM.

In an embodiment, the amount of IAM solution used herein is selected from about luL, about 2 μL, about 3 μL, about 4 μL, about 5 μL, about 6 μL, about 7 μL, about 8 μL, about 9 μL, about 10 μL, about 11 μL, about 12 μL, about 13 μL, about 14 μL, and about 15 μL.

In preferred embodiment, the amount of IAM solution used herein is selected from about 10 μL.

The term “electrokinetic injection” refers to the method to introduce sample in the capillary. Electrokinetic injection takes place when a voltage is applied to the electrode inside the sample solution driving the analytes to the capillary.

The term “injection duration” refers to the time required to inject the sample in the capillary during the process.

In an embodiment, the injection duration is selected from about 5 sec, about 10 sec, about 15 sec, about 20 sec, about 25 sec, about 30 sec, about 35 sec, about 40 sec, about 45 sec, and about 50 sec.

In preferred embodiment, the injection duration is selected from about 20 sec.

The term “voltage” refers to the voltage applied to the electrophoresis in which sample ions move under the influence of an applied voltage. The ion undergoes a force that is equal to the product of the net charge and the electric field strength. When the voltage is applied to the circuit, one electrode become net positive and the other net negative.

In an embodiment, the voltage applied to CE-SDS is selected from about 1 kV, about 2 kV, about 3 kV, about 4 kV, about 5 kV, about 6 kV, about 7 kV, about 8 kV, about 9 kV, about 10 kV, about 11 kV, about 12 kV, about 13 kV, about 14 kV, about 15 kV, about 16 kV, about 17 kV, about 18 kV, about 19 kV, and about 20 kV.

In an embodiment, the voltage applied to CE-SDS is selected from about 15 kV.

The term “migration run time” refers to the time it takes a solute to move from the beginning of the capillary to the detector window.

In an embodiment, the migration run time of the protein sample is selected from about 10 mins, about 20 mins, about 30 mins, about 40 mins, about 50 mins, about 60 mins, about 70 mins, about 80 mins, about 90 mins, and about 100 mins.

In preferred embodiment, the migration run time of the protein sample is selected from about 40 mins.

In an embodiment, the LMW impurities are separated at less than 22 minutes.

In another embodiment, the LMW impurities are separated at less than 20 minutes.

The term “temperature” refers to the temperature of capillary during the process. In an embodiment, the temperature of capillary is selected from about 10° C., about 15° C., about 20° C., about 25° C., about 30° C., about 35° C., about 40° C., about 45° C., and about 50° C.

In preferred embodiment, the temperature of capillary is selected from about 15° C., about 16° C., about 17° C., about 18° C., about 19° C., about 20° C., about 21° C., about 22° C., about 23° C., about 24° C. and about 25° C.

The term “partially purified” or “partially purification” are interchangeable and refers to the protein mixture that is obtained directly from harvest or from protein A chromatography column or from final purification or after any other chromatography column. In an embodiment, the protein mixture comprises LMW less than 0.5%. In another embodiment, the protein mixture comprises LMW less than about 0.3% to about 0.5%.

In an embodiment, the improved process for the separation of more than two low molecular weight proteins presents in the protein mixture comprising;
a) protein mixture comprising at least the impurity and the protein of interest, wherein the protein mixture does not contain a full-length antibody having molecular weight about 150 kDa;
b) mixing the protein mixture at a suitable ratio with sodium dodecyl sulphate or SDS to form treated protein mixture;
c) optionally incorporating a 10 kDa marker into the treated protein mixture;
d) injecting the treated protein mixture in CE-instrument;
e) performing CE-SDS by injecting the treated protein mixture in a CE-instrument; and
f) separating more than two low molecular weight proteins;
   wherein the treated protein mixture comprises protein to SDS ratio is higher than 2.7:1.

In an embodiment, the protein of interest is a fusion protein.

In another embodiment, the fusion protein is CTLA4-IgG having molecular weight less than 100 kda.

In certain embodiment, the composition of protein mixture comprising;
a) CTLA4-IgG fusion protein;
b) low molecular weight impurities comprises;
   i) first LMW about 80 kDa;
   ii) second LMW about 45 kDa;
   iii) third and fourth LMW about 25 kDa to 30 kDa;
      wherein the first, second, third and fourth LMWs are separated by CE-SDS.

In an embodiment, the treated protein mixture comprises protein to SDS ratio selected from about 3:1, about 3.2:1, about 3.3:1, about 3.4:1, about 3.5:1, about 3.6:1, about 3.7:1, about 3.8:1, about 3.9:1, about 4.0:1, about 4.1:1, about 4.2:1, about 4.3:1, about 4.4:1, about 4.5:1, about 4.6:1, about 4.7:1, about 4.8:1, about 4.9:1, about 5.0:1, and about 5:1.

In an embodiment, the protein concentration in treated protein mixture is selected from about 3 mg/ml, about 3.1 mg/ml, about 3.2 mg/ml, about 3.3 mg/ml, about 3.4 mg/ml, about 3.5 mg/ml, about 3.6 mg/ml, about 3.7 mg/ml, about 3.8 mg/ml, about 3.9 mg/ml, about 4.0 mg/ml, about 4.1 mg/ml, about 4.2 mg/ml, about 4.3 mg/ml, about 4.4 mg/ml and about 4.5 mg/ml.

In an embodiment, the separation and quantification of more than two low molecular weight impurity is improved compared to CE-SDS performed at protein concentration below 2.7 mg/ml.

In an embodiment, the separation and quantification of more than two low molecular weight impurity is improved compared to CE-SDS performed at protein concentration below 3.6 mg/ml.

In an embodiment, the separation and quantification of more than two low molecular weight impurity is improved compared to CE-SDS performed at protein concentration below 4.5 mg/ml.

In an embodiment, the separation and quantification of low molecular weight impurity is performed at 25° C.

In an embodiment, the CE-SDS is performed at suitable separation voltage to separate four low molecular weight protein in less than 22.

In an embodiment, the protein mixture is obtained from harvest or post affinity chromatography or post final purification steps.

In an embodiment, the main peak % is selected from about 97% to about 99%.

In an embodiment, the present invention provides an improved method for separation and quantification of impurities in a protein sample comprising;
a) obtaining a protein mixture from partially or completely purification comprising protein of interest and LMWs;
b) determine the amount of protein mixture about 270 μg;
c) mixing the protein mixture at a suitable ratio with sodium dodecyl sulphate or SDS to form treated protein mixture;
d) loading the treated protein mixture to said Capillary Electrophoresis-Sodium dodecyl sulfate (CE-SDS) capillary;
e) applying the suitable voltage to CE-SDS instrument;
f) separated and quantified the LMW species present in the treated protein mixture;
   wherein, separation and quantification of LMW is improved when protein amount loaded higher than 270 μg.

In an embodiment, the present invention provides an improved method for separation and quantification of impurities in a protein sample comprising;
a) obtaining a protein mixture from partially or completely purification comprising protein of interest and LMWs;
b) determine the amount of protein mixture about 360 μg;
c) mixing the protein mixture at a suitable ratio with sodium dodecyl sulphate or SDS to form treated protein mixture;
d) loading the treated protein mixture to said Capillary Electrophoresis-Sodium dodecyl sulfate (CE-SDS) capillary;
e) applying the suitable voltage to CE-SDS instrument;
f) separated and quantified the LMW species present in the treated protein mixture;
   wherein, separation and quantification of LMW is improved when protein amount loaded about or higher than 360 μg.

In an embodiment, the present invention provides an improved method for separation and quantification of impurities in a protein sample comprising;
a) obtaining a protein mixture from partially or completely purification comprising protein of interest and LMWs;
b) determine the amount of protein mixture about 360 μg;
c) mixing the protein mixture at a suitable ratio with sodium dodecyl sulphate or SDS to form treated protein mixture;
d) loading the treated protein mixture to said Capillary Electrophoresis-Sodium dodecyl sulfate (CE-SDS) capillary;
e) applying the suitable voltage to CE-SDS instrument;
f) separated and quantified the LMW species present in the treated protein mixture;
   wherein, separation and quantification of LMW is improved when protein amount loaded about or higher than 360 μg.

In an embodiment, the present invention provides an improved method for separation and quantification of impurities in a protein sample comprising;
a) obtaining a protein mixture from partially or completely purification comprising protein of interest and LMWs;
b) determine the amount of protein mixture about 450 μg;
c) mixing the protein mixture at a suitable ratio with sodium dodecyl sulphate or SDS to form treated protein mixture;
d) loading the treated protein mixture to said Capillary Electrophoresis-Sodium dodecyl sulfate (CE-SDS) capillary;
e) applying the suitable voltage to CE-SDS instrument;
f) separated and quantified the LMW species present in the treated protein mixture;
   wherein, separation and quantification of LMW is improved when protein amount loaded about or higher than 450 μg.

In an embodiment, the present invention provides an improved method for separation and quantification of impurities in a protein sample comprising;
a) obtaining a protein mixture from partially or completely purification comprising protein of interest and LMWs;
b) determine the amount of protein mixture about 450 μg;
c) mixing the protein mixture at a suitable ratio with sodium dodecyl sulphate or SDS to form treated protein mixture;
d) loading the treated protein mixture to said Capillary Electrophoresis-Sodium dodecyl sulfate (CE-SDS) capillary;
e) applying the suitable voltage to CE-SDS instrument;
f) separated and quantified the LMW species present in the treated protein mixture;
   wherein, separation and quantification of LMW is improved when protein amount loaded about or higher than 450 μg.

In an embodiment, the present invention provides an improved method for separation and quantification of impurities in a protein sample comprising;
a) obtaining a protein mixture from partially or completely purification comprising protein of interest and LMWs;
b) determine the amount of protein mixture about 450 μg;
c) mixing the protein mixture at a suitable ratio with sodium dodecyl sulphate or SDS to form treated protein mixture;
d) loading the treated protein mixture to said Capillary Electrophoresis-Sodium dodecyl sulfate (CE-SDS) capillary;
e) applying the suitable voltage to CE-SDS instrument;
f) separated and quantified the LMW species present in the treated protein mixture;
   wherein, separation and quantification of LMW is improved when protein amount loaded about or higher than 450 μg.

In an embodiment, the present invention provides an improved method for separation and quantification of impurities in a protein sample comprising;
a) obtaining a protein mixture from partially or completely purification comprising protein of interest and LMWs;
b) determine the amount of protein mixture about 450 μg;
c) mixing the protein mixture at a suitable ratio with sodium dodecyl sulphate or SDS to form treated protein mixture;
d) loading the treated protein mixture to said Capillary Electrophoresis-Sodium dodecyl sulfate (CE-SDS) capillary;
e) applying the suitable voltage to CE-SDS instrument;
f) separated and quantified the LMW species present in the treated protein mixture;
   wherein, separation and quantification of LMW is improved when protein amount loaded about or higher than 450 μg.

The present invention provides an example for illustration purpose which should not be considered to limit the scope of the present invention with the described examples.

### Example 1

### Process for Separation and Quantification of Peak of Protein Mixture Comprising CTLA4-IgG1 Fusion Protein 90 μg & 180 μg

### Materials/Reagents Details:

IgG purity heterogeneity assay kit
SDS MW sample buffer
SDS MW gel buffer
Basic wash solution (0.1 N NaOH)
Acidic wash solution (0.1 N HCl)
Iodoacetamide (IAM)
Water (Milli-Q)
10 kDa internal standard
Bare fused silica capillary
Preassembled capillary cartridge
Centrifuge tubes

### Equipment Details:

Capillary electrophoresis system
Dry-bath
Centrifuge
Microcentrifuge
Vortex mixer

Sample Details: Abatacept 25 mg/ml stored at 2-8° C. in plastic container.

### Preparation of Reagents

250 mM IAM solution: Accurately weighed 46 mg of iodoacetamide in 1.5 ml centrifuge tube and added 1000 ul of water into it. Vortex and mixed well.

### Sample Preparation:

For sample preparation of 90 μL of protein, pipetted the 90 μL sample from 1 mg/mL stock solution.

Diluted the sample with 100 μL SDS-MW Sample Buffer. Then added the 10 μL of 250 mM IAM solution and 2 μL of 10 kDa internal marker to the sample buffer solution. Incubated the solution for 65° C. for 11-12 mins.

For sample preparation of 180 μg of protein, 90 μL sample is pipetted from 2 mg/mL stock solution. The sample is diluted with 100 μL SDS-MW Sample Buffer. Then 10 μL of 250 mM IAM solution and 2 μL of 10 kDa internal marker is added to the sample buffer solution. The solution is incubated for 65° C. for 11-12 mins.

### Electrophoresis Conditions:

| | |
|---|---|
| Capillary: | Bare-fused silica, 50 μm ID × 20.0 cm |
| | length till detection window |
| Detection wavelength: | 220 nm |
| Sample temperature: | 25° C. |
| Capillary temperature: | 25° C. |
| Sample injection: | 5 kV for 20 sec (reverse polarity) |
| Separation: | Reverse polarity at 15 kV |
| Migration run time: | 40 min |

The UV lamp is turned on to allow it to warm up for at least 30 minutes prior to experimentation.

A pre-assembled cartridge or a pre-cut capillary assembled in the cartridge can be used.

The electrodes, capillary ends, opening levers, and interface block is cleaned carefully with water by using lint free tissue paper.

The capillary cartridge is positioned over the cartridge interface block and carefully lower the cartridge into position. The clamp bar is lowered and tighten the knobs. The cartridge cover door is lowered.

Conditioning of capillary: Capillary is rinsed with 0.1N NaOH for 10 mins to clean the capillary surface. The capillary surface is then rinsed with 0.1N HCl for 5 mins to neutralize. Then the water is rinsed for 2 mins to remove the acid residue. Then it is rinsed with SDS MW Gel buffer for 10 mins to fill the capillary with SDS gel.

After the capillary tip is rinse cleaned with double distilled water. The sample is injected for 20 sec at 5 KV. Run the sample for 40 mins at 15 KV.

### Result:

Sample containing 90 μg and 180 μg of protein, no LMW peaks are observed.

### Example 2: Process for Separation and Quantification of Peak of Protein Mixture Comprising CTLA4-IgG1 Fusion Protein 270 μg and 360 μg

### Materials/Reagents Details:

IgG purity heterogeneity assay kit
SDS MW sample buffer
SDS MW gel buffer
Basic wash solution (0.1 N NaOH)
Acidic wash solution (0.1 N HCl)
Iodoacetamide (IAM)
Water (Milli-Q)
10 kDa internal standard
Bare fused silica capillary
Preassembled capillary cartridge
Centrifuge tubes

### Equipment Details:

Capillary electrophoresis system
Dry-bath
Centrifuge
Microcentrifuge
Vortex mixer

Sample Details: Abatacept 25 mg/ml stored at 2-8° C. in plastic container.

### Preparation of Reagents

250 mM IAM solution: Accurately weighed 46 mg of iodoacetamide in 1.5 ml centrifuge tube and added 1000 ul of water into it. Vortex and mixed well.

### Sample Preparation:

For sample preparation of 270 μg of protein, 90 μL sample is pipetted from 3 mg/mL stock solution. Sample is diluted with 100 μL SDS-MW Sample Buffer. Then 10 μL of 250 mM IAM solution and 2 μL of 10 kDa internal marker is added to the sample buffer solution. The solution is incubated for 65° C. for 11-12 mins.

For sample preparation of 360 μg of protein, 90 μL sample is pipetted from 3 mg/mL stock solution. Sample is diluted with 100 μL SDS-MW Sample Buffer. Then 10 μL of 250 mM IAM solution and 2 μL of 10 kDa internal marker is added to the sample buffer solution. The solution is incubated for 65° C. for 11-12 mins.

### Electrophoresis Conditions:

| | |
|---|---|
| Capillary: | Bare-fused silica, 50 μm ID × 20.0 cm |
| | length till detection window |
| Detection wavelength: | 220 nm |
| Sample temperature: | 25° C. |
| Capillary temperature: | 25° C. |
| Sample injection: | 5 kV for 20 sec (reverse polarity) |
| Separation: | Reverse polarity at 15 kV |
| Migration run time: | 40 min |

The UV lamp is turned on to allow it to warm up for at least 30 minutes prior to experimentation.

A pre-assembled cartridge or a pre-cut capillary assembled in the cartridge can be used.

The electrodes, capillary ends, opening levers, and interface block is cleaned carefully with water by using lint free tissue paper.

The capillary cartridge is positioned over the cartridge interface block and carefully lower the cartridge into position. The clamp bar is lowered and tighten the knobs. The cartridge cover door is lowered.

Conditioning of capillary: Capillary is rinsed with 0.1N NaOH for 10 mins to clean the capillary surface. The capillary surface is then rinsed with 0.1N HCl for 5 mins to neutralize. Then the water is rinsed for 2 mins to remove the acid residue. Then it is rinsed with SDS MW Gel buffer for 10mins to fill the capillary with SDS gel.

After the capillary tip is rinse cleaned with double distilled water. The sample is injected for 20 sec at 5 KV. Run the sample for 40 mins at 15 KV.

### Separation and Quantification of Molecular Weight Related Impurities and Purity Determination of Protein Mixture Containing CTLA4-IgG1 Fusion Protein.

In this example, change in protein amount (270 μg and 360 μg) in sample preparation and increase in protein loading on capillary by electrokinetic injection were studied.

### Result:

Sample containing 270 μg of protein, at least two LMW peaks were observed.

Sample containing 360 μg of protein, at least three LMW peaks were observed.

### Example 3

### Process for Separation and Quantification of Peak of Protein Mixture Comprising CTLA4-IgG1 Fusion Protein 450 μg

### Materials/Reagents Details:

IgG purity heterogeneity assay kit
SDS MW sample buffer
SDS MW gel buffer
Basic wash solution (0.1 N NaOH)
Acidic wash solution (0.1 N HCl)
Iodoacetamide (IAM)
Water (Milli-Q)
10 kDa internal standard
Bare fused silica capillary
Preassembled capillary cartridge
Centrifuge tubes

### Equipment Details:

Capillary electrophoresis system
Dry-bath
Centrifuge
Microcentrifuge
Vortex mixer

Sample Details: Abatacept 25 mg/ml stored at 2-8° C. in plastic container.

### Preparation of Reagents

250 mM IAM solution: Accurately weighed 46 mg of iodoacetamide in 1.5 ml centrifuge tube and added 1000 μl of water into it. Vortex and mixed well.

### Sample Preparation:

For sample preparation of 450 μg of protein, pipetted the 90 μL sample from 5 mg/mL stock solution. Diluted the sample with 100 μL SDS-MW Sample Buffer. Then added the 10 μL of 250 mM IAM solution and 2 μL of 10 kDa internal marker to the sample buffer solution. Incubated the solution for 65° C. for 11-12 mins.

### Electrophoresis Conditions:

| | |
|---|---|
| Capillary: | Bare-fused silica, 50 μm ID × 20.0 cm |
| | length till detection window |
| Detection wavelength: | 220 nm |
| Sample temperature: | 25° C. |
| Capillary temperature: | 25° C. |
| Sample injection: | 5 kV for 20 sec (reverse polarity) |
| Separation: | Reverse polarity at 15 kV |
| Migration run time: | 40 min |

Turned on the UV lamp to allow it to warm up for at least 30 minutes prior to experimentation.

A pre-assembled cartridge or a pre-cut capillary assembled in the cartridge can be used.

Cleaned the electrodes, capillary ends, opening levers, and interface block carefully with water by using lint free tissue paper.

Positioned the capillary cartridge over the cartridge interface block and carefully lower the cartridge into position. Lowered the clamp bar and tighten the knobs. Lowered the cartridge cover door.

Conditioning of capillary: Capillary was rinsed with 0.1N NaOH for 10 mins to clean the capillary surface. Then rinsed with 0.1N HCl for 5 mins to neutralize the capillary surface. Then the water rinse for 2 mins to remove the acid residue. Then rinsed with SDS MW Gel buffer for 10 mins to fill the capillary with SDS gel.

After rinse cleaned the capillary tip with double distilled water. Injected the sample for 20 sec at 5 KV. Run the sample for 40 mins at 15 KV.

### Separation and Quantification of Molecular Weight Related Impurities and Purity Determination of Protein Mixture Containing CTLA4-IgG1 Fusion Protein.

In this example, change in protein amount (450 μg) in sample preparation and increase in protein loading on capillary by electrokinetic injection were studied.

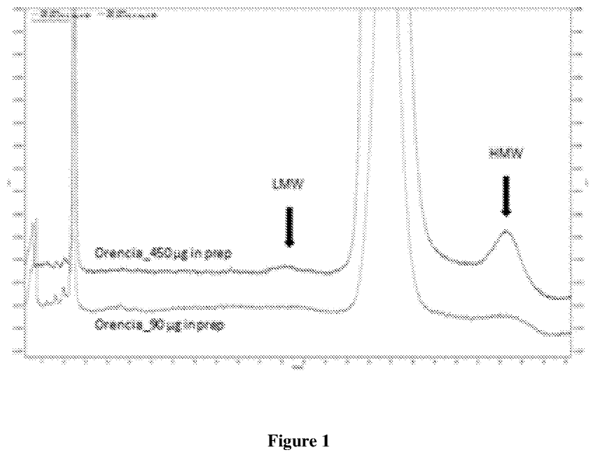

In Abatacept sample having higher protein (450 μg) amount, one peak of LMW impurity and HMW impurity was observed (FIG. 1) which were absent in sample having low protein amount (90 μg & 180 μg).

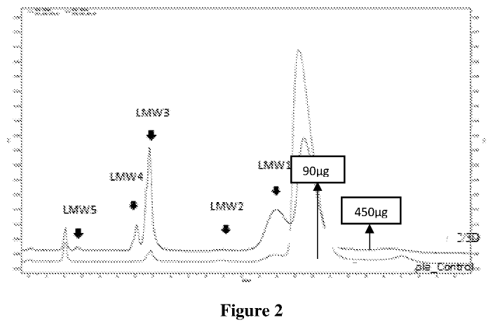

Different five LMW size variants were also observed in the sample containing 450 μg protein (FIG. 2).

### Result:

Higher amount of protein (450 μg) in sample preparation (from 5 mg/mL) helped to separate and quantify the impurities present in sample. Also approach of higher sample loading on capillary by increasing amount using concentration of 5 mg/ml was found significantly beneficial as compared to 90 μg, 180 μg, 270 μg and 360 μg protein in sample preparation. Therefore, 450 μg of protein amount during sample preparation (from 5 mg/mL) would be used for separation and analysis of impurities.

1. An improved process for the separation of more than two low molecular weight proteins presents in the protein mixture comprising;
a) protein mixture comprising at least the impurity and the protein of interest, wherein the protein mixture does not contain a full-length antibody having molecular weight about 150 kda;
b) mixing the protein mixture at a suitable ratio with sodium dodecyl sulphate or SDS to treated protein mixture;
c) optionally incorporating a 10 kDa marker into the treated protein mixture;
d) injecting the treated protein mixture in CE-instrument;
e) performing CE-SDS by injecting the treated protein mixture in a CE-instrument; and
f) separating more than two low molecular weight proteins;
wherein the treated protein mixture comprises protein to SDS ratio is higher than 2.7:1.

2. The process according to claim 1, wherein the protein of interest is fusion protein.

3. The process according to claim 2, wherein the fusion protein is CTLA4-IgG having molecular weight less than 100 kda.

4. A composition of protein mixture comprising;
a) CTLA4-IgG fusion protein;
b) low molecular weight impurities comprises;
i) first LMW about 80 kda;
ii) second LMW about 45 kda;
iii) third and fourth LMW about 25 kda to 30 kda;
wherein the first, second, third and fourth LMWs are separated by CE-SDS.

5. The process according to claim 1, wherein the treated protein mixture comprises protein to SDS ratio selected from about 3:1, about 3.6:1, about 4.5:1, and about 5:1.

6. The process according to claim 1, wherein the protein concentration in treated protein mixture is selected from about 3 mg/ml, about 3.5 mg/ml and about 4.5 mg/ml.

7. The process according to claim 1, wherein the separation or quantification of more than two low molecular weight impurity is improved compared to CE-SDS performed at protein concentration below 2.7 mg/ml.

8. The process according to claim 1, wherein the separation or quantification of more than two low molecular weight impurity is improved compared to CE-SDS performed at protein concentration below 3.6 mg/ml.

9. The process according to claim 1, wherein the separation or quantification of more than two low molecular weight impurity is improved compared to CE-SDS performed at protein concentration below 4.5 mg/ml.

10. The process according to claim 1, wherein the separation or quantification of low molecular weight impurity is performed at 25° C.

11. The process according to claim 1, wherein the CE-SDS is performed at suitable separation voltage to separate four low molecular weight protein in less than 22 minutes.

12. The process according to claim 1, wherein the protein mixture is obtained from harvest or post affinity chromatography or post final purification steps.
